(19) United States
(12) Patent Application Publication
(10) Pub. No.: US 2023/0166032 A1
(43) Pub. Date: Jun. 1, 2023

(54) CAPACITANCE SENSING FOR COMPONENT POSITIONING DETECTION

(71) Applicant: INSULET CORPORATION, Acton, MA (US)

(72) Inventors: Kepei SUN, Andover, MA (US); Benjamin LACROIX, Littleton, MA (US); Steven CARDINALI, Tewksbury, MA (US); Kyle BREINGAN, Lowell, MA (US); Jerry WITT, Andover, MA (US); Carolyn REISTAD, North Billerica, MA (US); Nicholas CONTE, Harvard, MA (US)

(21) Appl. No.: 18/059,170

(22) Filed: Nov. 28, 2022

Related U.S. Application Data
(60) Provisional application No. 63/284,150, filed on Nov. 30, 2021.

(51) Int. Cl. A61M 5/172

(52) U.S. Cl. CPC A61M 5/172

(57) ABSTRACT

Disclosed herein are drug delivery devices and methods for component positioning of a pump, such as a linear shuttle pump. In some approaches, a system may include first and second terminals movable with respect to one another, and a sensor device operable to detect a change in capacitance between the first and second terminals as the first and second terminals move with respect to one another. The sensor device may include a two-stage charger connected with a controller and a voltage source, the two-stage charger having a first capacitor connected with a first switch and a second capacitor connected with a second switch, the controller being operable to close the first switch to connect the first capacitor with the voltage source to charge the first capacitor, and open the first switch and close the second switch to connect the second capacitor with the voltage source to charge the second capacitor.

### RELATED APPLICATIONS

This application claims the benefit of U.S. Provisional Patent Application No. 63/284,150, filed Nov. 30, 2021, the contents of which are incorporated herein by reference in their entirety.

### FIELD

Embodiments herein generally relate to medication delivery. More particularly, embodiments herein relate to wearable drug delivery devices and methods for pump device component positioning detection using capacitance sensing.

### BACKGROUND

Many wearable drug delivery devices include a reservoir for storing a liquid drug and a drive mechanism, such as a pump including a pump chamber and piston, which is operated to expel the stored liquid drug from the reservoir for delivery to a user. A drawback with known devices is that the delivery rate accuracy suffers when the volume of liquid is small. Such inaccuracies arise in many cases from the drive mechanism(s) employed, which gives rise to variations in delivery rates. Accordingly, there is a need to provide a wearable drug delivery device capable of regulating drug delivery dosages while simultaneously verifying drive mechanism positioning and sequencing.

### SUMMARY

In some embodiments of the disclosure, a system may include a first terminal and a second terminal movable with respect to one another, and a sensor device operable to detect a change in capacitance between the first and second terminals as the first and second terminals move with respect to one another. The sensor device may include a two-stage charger connected with a controller and a voltage source, the two-stage charger including a first capacitor connected with a first switch and a second capacitor connected with a second switch, wherein the controller is operable to close the first switch to connect the first capacitor with the voltage source to charge the first capacitor, and to open the first switch and close the second switch to connect the second capacitor with the voltage source to charge the second capacitor.

In some embodiments of the present disclosure, a wearable drug delivery device may include a first terminal and a second terminal movable with respect to one another, wherein the first terminal is a part of a pump mechanism, and a sensor device operable to detect a change in capacitance between the first and second terminals as the first and second terminals move with respect to one another. The sensor device may include a two-stage charger connected with a controller and a voltage source, the two-stage charger comprising a first capacitor connected with a first switch and a second capacitor connected with a second switch, wherein the controller is operable to close the first switch to connect the first capacitor with the voltage source to charge the first capacitor, and to open the first switch and close the second switch to connect the second capacitor with the voltage source to charge the second capacitor.

In some embodiments of the present disclosure, a linear volume shuttle pump may include a first terminal and a second terminal movable with respect to one another, wherein the first terminal is a part of a pump mechanism, and a sensor device operable to detect a change in capacitance between the first and second terminals as the first and second terminals move with respect to one another. The sensor device may include a two-stage charger connected with a controller and a voltage source, the two-stage charger comprising a first capacitor connected with a first switch and a second capacitor connected with a second switch, wherein the controller is operable to close the first switch to connect the first capacitor with the voltage source to charge the first capacitor, and to open the first switch and close the second switch to connect the second capacitor with the voltage source to charge the second capacitor.

In some embodiments of the present disclosure, a method may include positioning a first terminal adjacent a second terminal, wherein the first terminal and the second terminal are movable with respect to one another, and detecting a change in capacitance between the first terminal and the second terminal using a sensor device, wherein the sensor device comprises a two-stage charger connected with a controller and a voltage source. The method may further include charging, by the controller, a first capacitor by closing a first switch to connect the first capacitor with the voltage source, and charging, by the controller, a second capacitor by opening the first switch and closing a second switch to connect the second capacitor with the voltage source.

### BRIEF DESCRIPTION OF DRAWINGS

The accompanying drawings illustrate example approaches of the disclosure, including the practical application of the principles thereof, as follows:

FIG. 1 illustrates a perspective view of an example linear volume shuttle fluid pump according to embodiments of the present disclosure;

FIG. 2 illustrates an end view of the linear volume shuttle fluid pump depicted in FIG. 1 according to embodiments of the present disclosure;

FIGS. 3A-3B are simplified representations of a first terminal and a second terminal during use, according to embodiments of the present disclosure;

FIG. 4 is a schematic of a sensor device according to embodiments of the present disclosure;

FIGS. 5A-5B are graphs illustrating continuous charging voltage curves according to embodiments of the present disclosure;

FIG. 6 is a schematic of a sensor device according to embodiments of the present disclosure;

FIGS. 7A-7B are graphs illustrating continuous charging voltage curves according to embodiments of the present disclosure;

FIG. 8 illustrates a process according to embodiments of the present disclosure;

FIG. 9 illustrates a process according to embodiments of the present disclosure; and

FIG. 10 illustrates a schematic diagram of a drug delivery system according to embodiments of the present disclosure.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, some reference numbers may be omitted in certain drawings.

### DETAILED DESCRIPTION

Various approaches in accordance with the present disclosure will now be described more fully hereinafter with reference to the accompanying drawings, where embodiments of the methods are shown. The approaches may be embodied in many different forms and are not to be construed as being limited to the embodiments set forth herein. Instead, these embodiments are provided so this disclosure will be thorough and complete, and will fully convey the scope of the approaches to those skilled in the art.

Various examples disclosed herein provide a drive mechanism and/or pump system with the ability to control and more accurately verify pump position and sequencing. As a result, a drug delivery device that contains a reservoir and a pump may be made more reliable and thus safer for users.

Various examples described herein enable a pump, such as a linear volume shuttle pump (LVSP), to execute a pumping cycle in a proper sequence. At any given time during pump actuation, it is beneficial to know the location of different pump components, namely a pump chamber and a piston, as the pump chamber and the piston are responsible for drawing in and expelling fluid. Knowing the location of both the pump chamber and the piston also indicates whether the pump operates in a designed sequence. In some examples of the present disclosure, a first terminal or contact may be located on a piston grip coupled to the piston, and a second terminal or contact may be positioned below the piston grip. In some examples, the second terminal may be a conductive component or plate (e.g., copper), which is coupled to or embedded within or on top of a printed circuit board (PCB). As will be described in greater detail herein, the capacitance formed between the two terminals can be used to detect the position of the moving mechanical parts of the pump, which in turn can be used for tracking the location of the chamber and the piston. The position information obtained by the system about the position of the pump chamber can be used, for example, to ensure that the pump is drawing in or expelling fluid appropriately, and ultimately the volume of fluid drawn in or expelled.

Although described herein in the context of a LVSP, other types of pump mechanisms for a wearable liquid delivery device are possible within the scope of the present disclosure. Furthermore, the wearable drug delivery device described herein may include an analyte sensor, such as a blood glucose sensor, and a cannula or microneedle array of the sensor(s) may be operable in allowing the device to measure an analyte level in a user of the device.

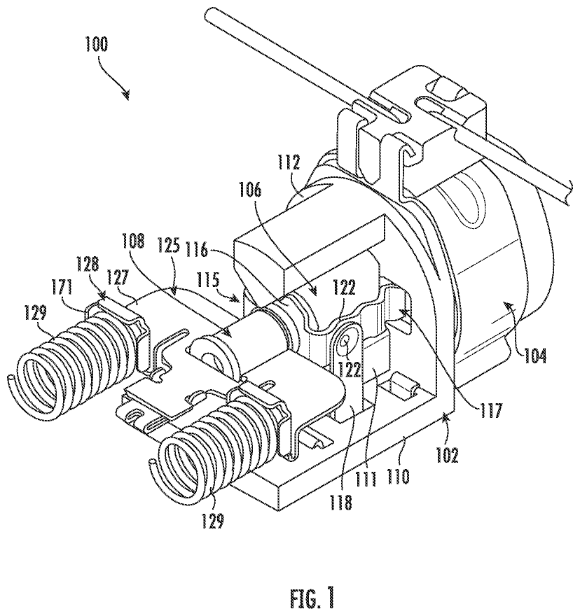

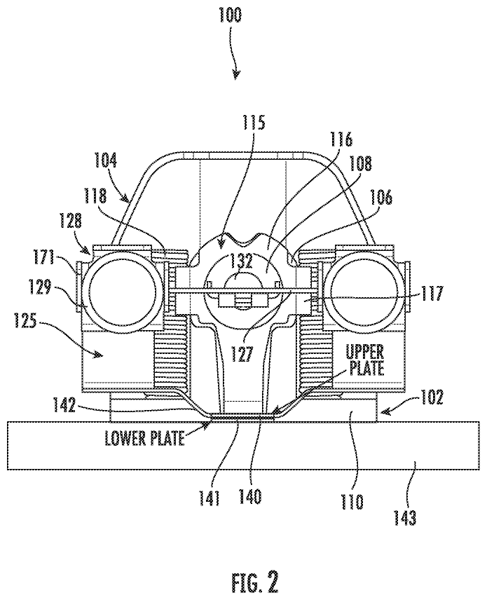

FIGS. 1-2 illustrate a LSVP 100 (hereinafter “pump”) according to embodiments of the present disclosure. As shown, the pump 100 may include a pump housing 102 coupling together a fluid reservoir 104, a pump chamber 106, and a piston 108. In the position demonstrated, the piston 108 may be full inserted within the pump chamber 106, at the end of its stroke. In some embodiments, the fluid reservoir 104 may contain a fluid or liquid drug. The pump housing 102 may include a base 110, a chassis 111 extending from the base 110 for retaining the pump chamber 106, and a reservoir wall 112 operable to interface with the pump chamber 106. Although non-limiting, the pump housing 102 may be formed from an injection molded plastic or other similar material.

Although not shown, the pump chamber 106 may include an inlet pathway or component and an outlet pathway or component. A liquid or fluid can enter the pump chamber 106 through the inlet pathway and can exit the pump chamber 106 through the outlet pathway. One or more plunger components may operate with the inlet and outlet pathways to draw a fluid into the pump chamber 106 and to expel the fluid from the pump chamber 106. In various examples, the pump chamber 106 may be coupled to the fluid reservoir 104 that stores a fluid or liquid drug. For example, the inlet may be coupled to the fluid reservoir 104 and the outlet pathway may be coupled to a fluid path component (not shown) that is coupled to a patient or user that is to receive the liquid drug stored in the fluid reservoir 104.

As further shown, the pump 100 may include a detent apparatus 115 coupled to the pump chamber 106. In some embodiments, the detent apparatus 115 may include a detent cap or body 116, one or more detent arms 117 extending from the detent body 116, and one or more detent engagement members 118. As shown, the detent engagement members 118 may extend from the base 110. The detent body 116 may extend over and/or abut one end of the pump chamber 106. In some embodiments, the detent body 116 may further abut the piston 108, wherein an opening (not shown) of the detent body 116 may allow a rod 132 (FIG. 2) of the piston 108 to pass therethrough. It will be appreciated that the detent apparatus 115 is non-limiting, and that other pump structures are possible within the scope of the present disclosure.

The detent arms 117 may include one or more arrest locations, which may be recesses or valleys disposed between one or more peaks. The arrest locations may be curved to generally compliment the dimensions of the detent engagement member 118, which in this case, may include a rounded protrusion 122. The arrest locations may allow discrete positioning of the pump chamber 106 and/or the piston 108 by adding additional frictional forces to restrict movement of the detent body 116 prior to a desired time.

As further shown, the pump 100 may include a piston grip 125 coupled to the piston 108. The piston grip 125 may include one or more grip components (not shown) engaged with an exterior of the piston 108. During operation, movement of the piston grip 125 causes the piston 108 to move axially relative to the pump chamber 106 to control receipt and delivery of a liquid within the pump chamber 106. The piston grip 125 may be actuated by a variety of mechanisms and/or actuators. In various examples, the piston grip 125 may be actuated by an actuator capable of producing reciprocating motion, for example, a piezoelectric-based actuator, a solenoid-based actuator, a Nitinol-based actuator, a spring-based actuator, a rotary motor with a gear train, a direct current (DC) motor, or any combination thereof. With each of these examples, a desired effect of shuttling fluid may be achieved.

In some embodiments, the piston grip 125 may include a grip body 127 extending on opposite sides of the piston 108. The grip body 127 may be a generally planar component including one or more spring footers 128 extending therefrom. As shown, each spring footer 128 may include one or more tabs 171 to engage and retain therein a side spring 129. In this embodiment, two side springs 129 may be disposed on opposite sides of the piston 108, parallel to a central axis extending through the piston 108, the pump chamber 106, and the detent body 116, though one spring may be used in alternate embodiments, and may be in axial alignment with piston 108. The side springs 129 may provide a spring force to bias the piston grip 125, and thus the piston 108, towards the pump chamber 106, or in other embodiments, away from the pump chamber 106.

As shown in FIG. 2, in some embodiments, the pump 100 may include a first terminal 140 coupled to, or part of, the piston grip 125, or otherwise moveable with piston 108. More specifically, the first terminal 140 may be a conductive plate (e.g., copper) coupled to a lower bridge 142 of the piston grip 125. The lower bridge 142 may extend over a second terminal 141, which may also be a conductive plate (e.g., copper) coupled to or embedded within a PCB 143. The first terminal 140 may be secured to a number of different portions of piston grip 125 in alternative embodiments.

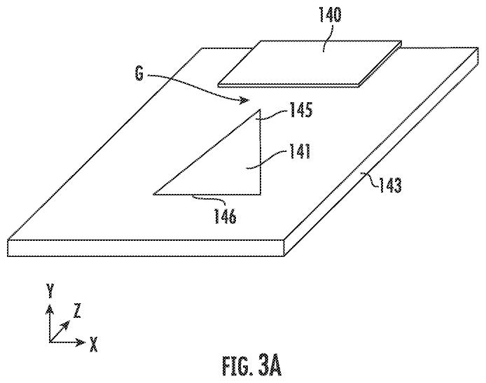

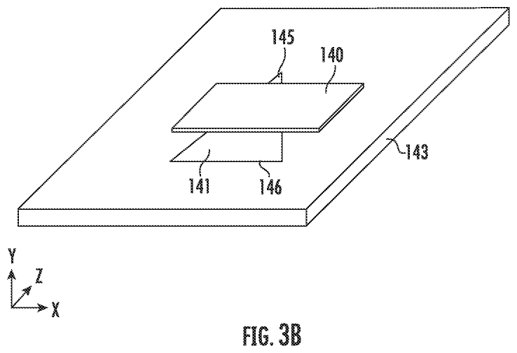

FIGS. 3A-3B, are simplified representations of the first terminal 140, the second terminal 141, and a substrate (e.g., the PCB 143) during use. The first terminal 140 and the piston grip (not shown) may travel in a reciprocal fashion between a first position, shown in FIG. 3A, and a second position, shown in FIG. 3B. In some embodiments, the second terminal 141 has a varied shape (e.g., triangle), which causes the capacitance to increase between a first end 145 and a second end 146 of the second terminal 141 due to the increased surface area overlap between the first and second terminals 140, 141. Between the first and second terminals 140, 141, a spacing distance (e.g., in the y-direction) may be selected to form a detectable range of capacitance. Although non-limiting, the spacing distance may be between 50-200 microns. In some embodiments, the substrate is a dielectric, and a smaller space or gap ‘G’ (FIG. 3A) between the first and second terminals 140, 141 may be provided if a higher capacitance is desired and the added friction does not impact motion between the first and second terminals 140, 141.

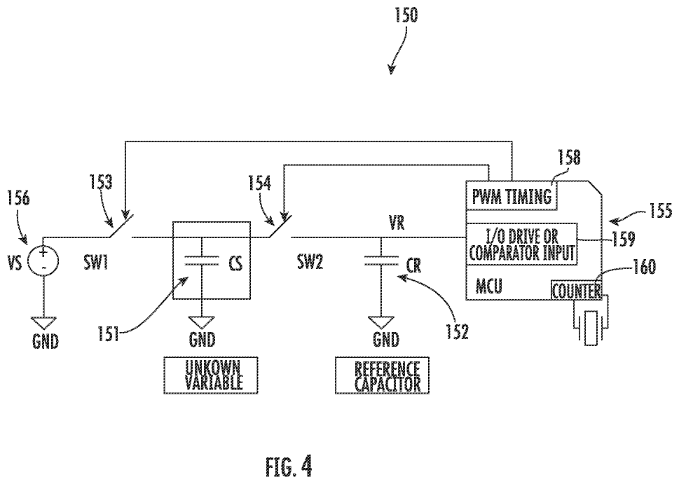

FIG. 4 is a schematic of a sensor device 150 operable to detect a change in capacitance between the first and second terminals 140, 141 as the first and second terminals 140, 141 move with respect to one another. As shown, the sensor device 150 may be a two-stage charger including a first capacitor (CS) 151, a second capacitor 152 (CR), a first switch (SW1) 153, and a second switch 154 (SW2). The first and second capacitors 151, 152 may be connected on one side to a voltage source (VS) 156 and on a second side to a controller 155, which may be a microcontroller unit (MCU). Although non-limiting, the controller 155 may include a pulse width modulation (PWM) timer 158, an input/output drive or comparator input 159, and a counter 160. The first switch 153 may be located between the first capacitor 151 and the voltage source 156, while the second switch 154 may be located between the first capacitor 151 and the second capacitor 152.

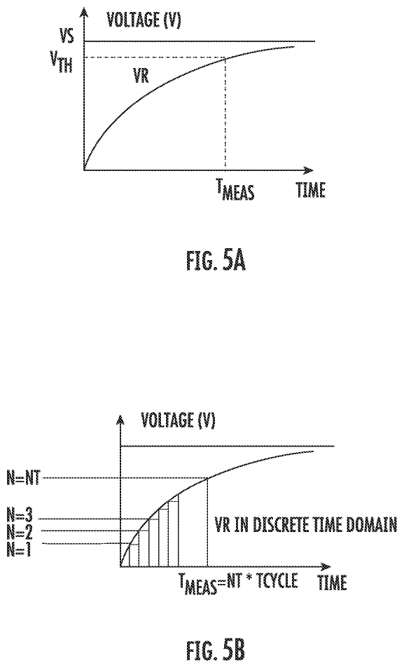

During use, the controller 155 may operate the first and second switches 153, 154 to charge the voltage on the second capacitor 152. For example, for each filling and dispensing cycle of the pump chamber 106, the controller 155 may connect the voltage source 156 with the first capacitor 151 to fully charge the first capacitor 151, and then open the first switch 153 and close the second switch 154 to equalize the voltages of the first and second capacitors 151, 152. The voltage of the second capacitor 152 may appear as a rising, continuous charging curve, as shown in FIG. 5A. FIG. 5B demonstrates a charging curve over discreet time periods. In some embodiments, the charging process could be viewed as a discrete time pumping process, and with each nth time from a fully charged VS by the voltage source 156, the rise of the VR could be calculated according to equation 1, as follows:

$$V {R}_{n} = V {R}_{n - 1} + ( V S - V {R}_{n - 1} ) \frac{CS}{C S + C R} \qquad ( 1 )$$

In some embodiments, the measurement duration Tₘₑₐₛ may be obtained by the controller 155 by counting the incrementing counter value from the start of the charging of VR, until the VR reaches a certain threshold. The threshold may either be the digital I/O level ‘High,’ or a voltage trigger value set in the input of the comparator 159.

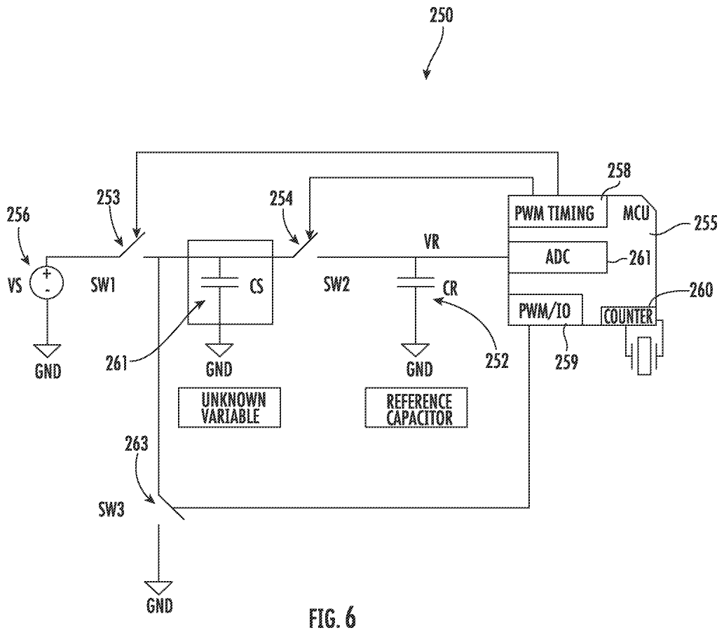

FIG. 6 is a schematic of a sensor device 250 operable to detect a change in capacitance between first and second terminals as the first and second terminals move with respect to one another. In this embodiment, a Kalman filter may be employed to enable fast detection of charging/discharging of a first capacitor (Cs) 251 and/or a second capacitor 252 (Cr). As shown, the sensor device 250 may further include a first switch (SW1) 253 and a second switch 254 (SW2). The first and second capacitors 251, 252 may be connected on one side to a voltage source (Vs) 256 and on a second side to a controller 255, which may be an MCU. Although non-limiting, the controller 255 may include a PWM timer 258, a PWM/IO 259, a counter 260, and an application delivery controller (ADC) 261. The first switch 253 may be located between the first capacitor 251 and the voltage source 256, while the second switch 254 may be located between the first capacitor 251 and the second capacitor 252. In this embodiment, the sensor device 250 may further include a third switch (SW3) 263.

Using the Kalman filter, the second capacitor 252 may be charged and discharged continuously, as the first switch 253 is used in a charge phase and the third switch 263 is used in a discharge phase. As motion of the first and/or second terminal occurs, capacitance of the first capacitor 251 changes abruptly, whereas the estimated capacitance Cs in the Kalman filter changes after a few samples being read in ADC. Therefore, within a minimum amount of delay, the motion of the piston grip could be captured by the sensor device 250. Said another way, the delay of motion being sensed (i.e., motion of the first and/or second terminals relative to each other) is minimized using the Kalman filter.

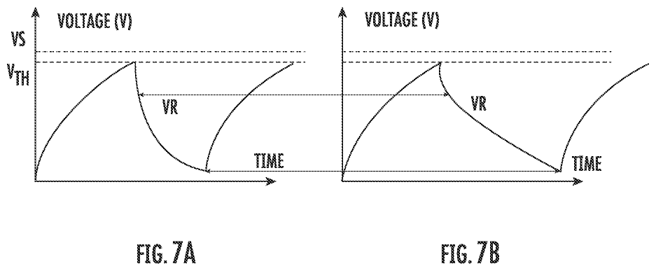

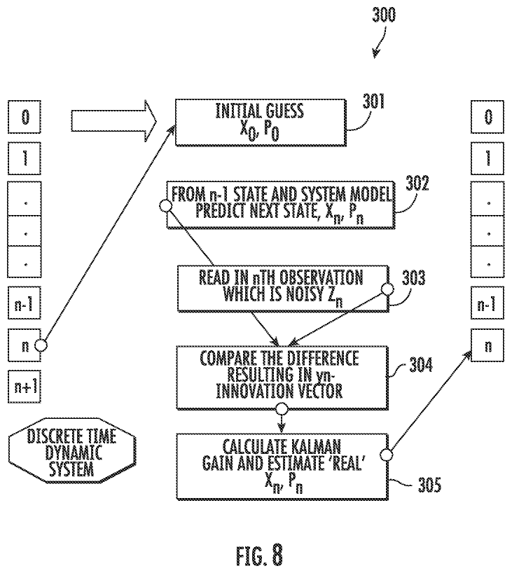

In some embodiments, the discharge of the second capacitor 252 may be unnecessary. The voltage (VR) curve can be demonstrated in FIGS. 7A-7B, wherein FIG. 7A shows the continuous charge/discharge of the first capacitor 251 with no motion, and FIG. 7B shows the continuous charge/discharge of the first capacitor with motion occurring in the middle of the charge/discharge. When the mechanical motion of the terminals and the capacitance sensing are not synchronized, this continuous operation would allow the ‘instant’ detection of the motion. Without continuous operation, the measurement of the capacitance could only be done at certain intervals, and discharge of the second capacitor 252 would lead to mis-detection. However, using the Kalman filter, the first capacitor 251 may be charged and discharged continuously.

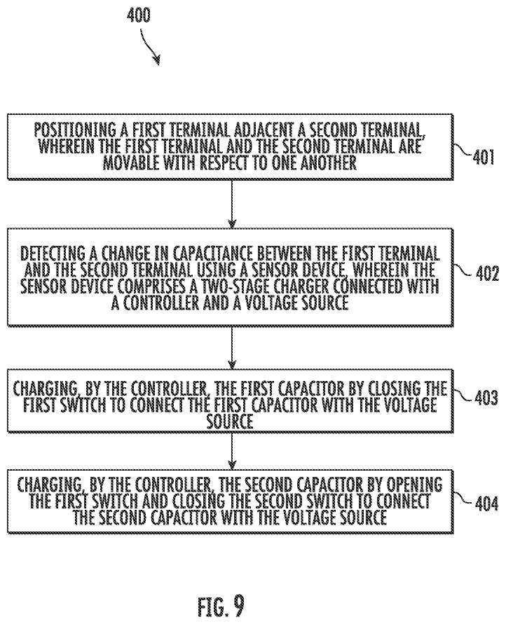

Referring to FIG. 8, a process 300 using the Kalman filter according to embodiments of the present disclosure will be described in greater detail. The Kalman filter is a time-domain filter which requires minimal memory space for storage of historical data. The Kalman filter continuously estimates what the next voltage point and the actual capacitance value is. In some embodiments, an Extended Kalman Filter (EKF) model is used, wherein the algorithm of the Kalman filter uses an estimation of voltage change and an estimate of the capacitor value at the same time, resulting in the implementation of a simultaneous parameter and system state estimation design.

More specifically, at block 301, an initial prediction of the voltage (VR) at t−0 is performed, according to the following equation:

VR₀=0 (2)

The EKF result may not be sensitive to the initial guess values, although in some embodiments,

X₀=E[X] (3)

where X is the variable or state to estimate.

In some embodiments, in which the simulation uses a 3 pF capacitor in the curve generation, the initial prediction of CS for the first capacitor 251 is calculated according to the following equation:

CS₀=1pF (4)

The initial guess forms a vector containing the state VR and parameter CS to estimate as follows:

$${X}_{0} = [ \begin{matrix}V {R}_{0} \\ {CS}_{0}\end{matrix} ] \qquad ( 5 )$$

In some embodiments, the initial guess may also include the variances of the two variables. The covariance matrix P may be initialized, as shown by the following equation:

$${P}_{0} = [ \begin{matrix}0 & 0 \\ 0 & {σ}_{c}^{2}\end{matrix} ] \qquad ( 6 )$$

Next, at block 302, the next variable values (e.g., Xn and Pn) are predicted. In some embodiments, the estimation may be based on the system modeling as follows:

$${\tilde{X}}_{n} = [ \begin{matrix}V {R}_{n - 1}^{+} + ( V S - V {R}_{n - 1} ) \frac{{CS}_{n - 1}^{+}}{C {S}_{n - 1}^{+} + C R} \\ {CS}_{n - 1}^{+}\end{matrix} ] \qquad ( 7 )$$

Along with the state and parameter prediction, the covariance matrix is predicted as follows:

Pₙ⁻=Fₙ₋₁Pₙ₋₁⁺Fₙ₋₁^{T}+Q (8)

In this case, Fₙ₋₁ is the Jacobian matrix of the state/parameter transition matrix derived from equation (7), and Q is the covariance matrix for F. The prediction is the controller's ‘guess’ of what the next state would be.

Next, at block 303, the predicted values then will be compared with the observations which is defined as:

$${Z}_{n} = [ \begin{matrix}V {R}_{n}^{A D C} \\ CR \frac{{VR}_{n}^{-} - {VR}_{n - 1}^{+}}{VC - {VR}_{n}^{-}}\end{matrix} ] \qquad ( 9 )$$

The superscript ADC indicates that the value is the read-in value of the ADC 261. Different methods for modeling the observations Zn can be performed in other embodiments. After the observation is calculated, at block 304 the difference (i.e., innovation vector) is calculated as follows:

yₙ=Zₙ−\tilde{X}ₙ (10)

At block 305, Kalman Gain may be consequently calculated as follows:

Kₙ=Pₙ⁻Hₙ^{T}(R+HₙPₙ⁻Hₙ^{T})⁻¹ (11)

In this case, Hₙ is the Jacobian matrix of the state observation vector (9). With the Kalman Gain calculated, the estimation of the ‘updated-by-observation’ results may be found as follows:

Xₙ⁺=\tilde{X}ₙ+Kₙ·yₙ (12)

Lastly, the covariance matrix associated with the updated estimation is calculated as follows:

Pₙ⁺=(I−KₙHₙ)Pₙ⁻ (13)

In an alternative embodiment, the process 300 could ignore the voltage estimation (equation (2)) and use the ADC read-in values for observation only, and focus on the estimation of CS only. This method would allow the convergence of capacitance estimation faster.

In some embodiments, the process 300 can be further improved to a more stable implementation and faster convergence once the algorithm's actual data from the MCU reading are available and the computation is executed in the MCU 255. The closer the model is to real system behaviors, the faster the algorithm will converge.

Turning now to FIG. 9, another process 400 according to embodiments of the present disclosure will be described. At block 401, the process may include positioning a first terminal adjacent a second terminal, wherein the first terminal and the second terminal are movable with respect to one another. In some embodiments, the first terminal is part of, or coupled to, a piston grip of a wearable drug delivery device. The second terminal may be part of a substrate (e.g., PCB) beneath the piston grip. In some embodiments, the second terminal is a conductive plate having a varied geometry from a first end to a second end to create a correspondingly varied capacitance as the first and second terminals move relative to one another.

At block 402, the process 400 may include detecting a change in capacitance between the first terminal and the second terminal using a sensor device, wherein the sensor device comprises a two-stage charger connected with a controller and a voltage source. In some embodiments, the sensor device may include a first capacitor, a second capacitor, a first switch, and a second switch. In some embodiments, the sensor device may include a third switch. The first and second capacitors may be connected on one side to a voltage source and on a second side to a controller, which may be a microcontroller unit. The first switch may be located between the first capacitor and the voltage source, while the second switch may be located between the first capacitor and the second capacitor. In some embodiments, a Kalman filter may be employed to enable fast detection of charging/discharging of a first capacitor and/or a second capacitor.

At block 403, the process 400 may include charging, by the controller, the first capacitor by closing the first switch to connect the first capacitor with the voltage source. At block 404, the process 400 may include charging, by the controller, the second capacitor by opening the first switch and closing the second switch to connect the second capacitor with the voltage source.

In some embodiments, the process 400 may further include equalizing, by the controller, a first voltage of the first capacitor and a second voltage of the second capacitor for each pumping cycle. In some embodiments, the process 400 may further include continuously charging and discharging the second capacitor using a Kalman filter. In some embodiments, continuously charging and discharging the second capacitor may include opening, by the controller, a third switch when the first capacitor is being charged, and closing, by the controller, the third switch when the first capacitor is being discharged.

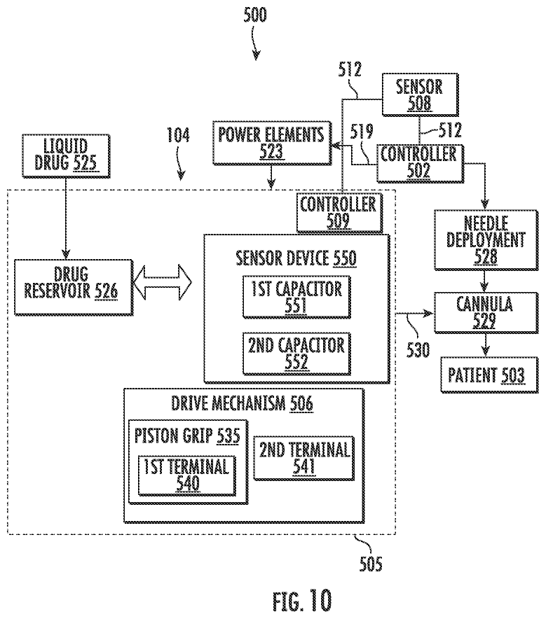

FIG. 10 illustrates a simplified block diagram of an example system (hereinafter “system”) 500. The system 500 may be a wearable or on-body drug delivery device and/or an analyte sensor attached to the skin of a patient 503. The system 500 may include a controller 502, a pump mechanism 504 (hereinafter “pump 504”), and a sensor 508. The sensor 508 may be a glucose or other analyte monitor such as, for example, a continuous glucose monitor, and may be incorporated into the wearable device. The sensor 508 may, for example, be operable to measure blood glucose (BG) values of a user to generate a measured BG level signal 512. The controller 502, the pump 504, and the sensor 508 may be communicatively coupled to one another via a wired or wireless communication path. For example, each of the controller 502, the pump 504 and the sensor 508 may be equipped with a wireless radio frequency transceiver operable to communicate via one or more communication protocols, such as Bluetooth®, or the like. The system 500 may also include a delivery pump device (hereinafter “device”) 505, which includes a drive mechanism 506 coupled to a reservoir 526 for driving a liquid drug 525 therefrom. In some embodiments, the drive mechanism 506 may include a first terminal 540 coupled to, or part of, a piston grip 535. In some embodiments, the first terminal 540 may be a conductive plate (e.g., copper) coupled to a lower bridge of the piston grip 535. The lower bridge may extend over a second terminal 541, which may also be a conductive plate (e.g., copper) coupled to or embedded within a PCB (not shown). The system 500 may include additional components not shown or described for the sake of brevity.

The controller 502 may receive a desired BG level signal, which may be a first signal, indicating a desired BG level or range for the patient 503. The desired BG level signal may be stored in memory of a controller 509 on device 505, received from a user interface to the controller 502, or another device, or by an algorithm within controller 509 (or controller 502) that automatically determines a BG level for the patient 503. The sensor 508 may be coupled to the patient 503 and operable to measure an approximate value of a BG level of the patient 503. In response to the measured BG level or value, the sensor 508 may generate a signal indicating the measured BG value. As shown, the controller 502 may also receive from the sensor 508 via a communication path, the measured BG level signal 512, which may be a second signal.

Based on the desired BG level signal and the measured BG level signal 512, the controller 502 or controller 509 may generate one or more control signals for directing operation of the pump 504. For example, one control signal 519 from the controller 502 or controller 509 may cause the pump 504 to turn on, or activate one or more power elements 523 operably connected with the device 505. The specified amount of the liquid drug 525 may be determined as an appropriate amount of insulin to drive the measured BG level of the user to the desired BG level. Based on operation of the pump 504, as determined by the control signal 519, the patient 503 may receive the liquid drug from the reservoir 526. The system 500 may operate as a closed-loop system, an open-loop system, or as a hybrid system. In an exemplary closed-loop system, the controller 509 may direct operation of the device 505 without input from the controller 502, and may receive BG level signal 512 from the sensor 508. The sensor 508 may be housed within the device 505 or may be housed in a separate device and communicate wirelessly directly with the device 505.

As further shown, the system 500 may include a needle deployment component 528 in communication with the controller 502 or the controller 509. The needle deployment component 528 may include a needle/cannula 529 deployable into the patient 503 and may have one or more holes at a distal end thereof. The device 505 may be connected to the needle/cannula 529 by a fluid path component 530. The fluid path component 530 may be of any size and shape and may be made from any suitable material. The fluid path component 530 can allow fluid, such as the liquid drug 525 in the reservoir 526, to be transferred to the needle/cannula 529.

The controller 502/509 may be implemented in hardware, software, or any combination thereof. The controller 502/509 may, for example, be a processor, a logic circuit or a microcontroller coupled to a memory. The controller 502/509 may maintain a date and time as well as other functions (e.g., calculations or the like) performed by processors. The controller 502/509 may be operable to execute an artificial pancreas (AP) algorithm stored in memory (not shown) that enables the controller 502/509 to direct operation of the pump 504. For example, the controller 502/509 may be operable to receive an input from the sensor 508, wherein the input indicates an automated insulin delivery (AID) application setting. Based on the AID application setting, the controller 502/509 may modify the behavior of the pump 504 and resulting amount of the liquid drug 525 to be delivered to the patient 503 via the device 505.

In some embodiments, the controller 502/509 may operate with a sensor device 550, which may be same or similar to the sensor device 150 or the sensor device 250 described above. The sensor device 550 may be part of the device 505, as shown, or located external to the device 505. In some embodiments, the sensor device 550 may be a two-stage charger including a first capacitor 551 and a second capacitor 552. The first and second capacitors 551, 552 may be connected on one side to a voltage source, such as the power element(s) 523, and on a second side to the controller 502/509. During use, the controller 502/509 may operate first and second switches of the sensor device 550 to charge up the voltage on the second capacitor 552. For example, for each filling and dispensing cycle of the pump chamber, the controller 502/509 may connect the voltage source with the first capacitor 551 to fully charge the first capacitor 551, and then open the first switch and close the second switch to equalize the voltages of the first and second capacitors 551, 552. The voltage of the second capacitor 552 may appear as a rising, continuous charging curve.

In some embodiments, using a Kalman filter, the second capacitor 552 may be charged and discharged continuously, as the first switch is used in a charge phase and a third switch is used in a discharge phase. As motion of the first terminal 540 and/or second terminal 541 occurs, capacitance of the first capacitor 551 changes abruptly, whereas the estimated capacitance in the Kalman filter changes after a few samples being read in an ADC of the controller 502/509. Therefore, within a minimal amount of delay (e.g., 100-400 μs), the motion of the piston grip 535 could be captured by the sensor device 550. Said another way, the delay from motion starting to the motion being sensed is minimized using the Kalman filter.

In some embodiments, the sensor 508 may be, for example, a continuous glucose monitor (CGM). The sensor 508 may be physically separate from the pump 504, or may be an integrated component within a same housing thereof. The sensor 508 may provide the controller 502 with data indicative of measured or detected blood glucose levels of the user.

The power element 523 may be a battery, a piezoelectric device, or the like, for supplying electrical power to the device 505. In other embodiments, the power element 523, or an additional power source (not shown), may also supply power to other components of the pump 504, such as the controller 502, memory, the sensor 508, and/or the needle deployment component 528.

In an example, the sensor 508 may be a device communicatively coupled to the controller 502 and may be operable to measure a blood glucose value at a predetermined time interval, such as approximately every 5 minutes, 10 minutes, or the like. The sensor 508 may provide a number of blood glucose measurement values to the AP application.

In some embodiments, the pump 504, when operating in a normal mode of operation, provides insulin stored in the reservoir 526 to the patient 503 based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the sensor 508 or other functional elements of the pump 504. For example, the pump 504 may contain analog and/or digital circuitry that may be implemented as the controller 502/509 for controlling the delivery of the drug or therapeutic agent. The circuitry used to implement the controller 502/509 may include discrete, specialized logic and/or components, an application-specific integrated circuit, a microcontroller or processor that executes software instructions, firmware, programming instructions or programming code enabling, for example, an AP application stored in memory, or any combination thereof. For example, the controller 502/509 may execute a control algorithm and other programming code that may make the controller 502/509 operable to cause the pump to deliver doses of the drug or therapeutic agent to a user at predetermined intervals or as needed to bring blood glucose measurement values to a target blood glucose value. The size and/or timing of basal and/or bolus doses may be determined automatically based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like), or may be pre-programmed, for example, into the AP application by the patient 503 or by a third party (such as a health care provider, a parent or guardian, a manufacturer of the wearable drug delivery device, or the like) using a wired or wireless link.

Although not shown, in some embodiments, the sensor 508 may include a processor, memory, a sensing or measuring device, and a communication device. The memory may store an instance of an AP application as well as other programming code and be operable to store data related to the AP application.

In various embodiments, the sensing/measuring device of the sensor 508 may include one or more sensing elements, such as a blood glucose measurement element, a heart rate monitor, a blood oxygen sensor element, or the like. The sensor processor may include discrete, specialized logic and/or components, an application-specific integrated circuit, a microcontroller or processor that executes software instructions, firmware, programming instructions stored in memory, or any combination thereof.

The foregoing discussion has been presented for purposes of illustration and description and is not intended to limit the disclosure to the form or forms disclosed herein. For example, various features of the disclosure may be grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein.

The phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other.

Furthermore, identification references (e.g., primary, secondary, first, second, third, fourth, etc.) are not intended to connote importance or priority but are used to distinguish one feature from another. The drawings are for purposes of illustration only and the dimensions, positions, order and relative sizes reflected in the drawings attached hereto may vary.

Furthermore, the terms “substantial” or “substantially,” as well as the terms “approximate” or “approximately,” can be used interchangeably in some embodiments, and can be described using any relative measures acceptable by one of ordinary skill in the art. For example, these terms can serve as a comparison to a reference parameter, to indicate a deviation capable of providing the intended function. Although non-limiting, the deviation from the reference parameter can be, for example, in an amount of less than 1%, less than 3%, less than 5%, less than 10%, less than 15%, less than 20%, and so on.

Still furthermore, although the various methods disclosed herein are described as a series of acts or events, the present disclosure is not limited by the illustrated ordering of such acts or events unless specifically stated. For example, some acts may occur in different orders and/or concurrently with other acts or events apart from those illustrated and/or described herein, in accordance with the disclosure. In addition, not all illustrated acts or events may be required to implement a methodology in accordance with the present disclosure. Furthermore, the methods may be implemented in association with the formation and/or processing of structures illustrated and described herein as well as in association with other structures not illustrated.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, the present disclosure has been described herein in the context of a particular implementation in a particular environment for a particular purpose. Those of ordinary skill in the art will recognize the usefulness is not limited thereto and the present disclosure may be beneficially implemented in any number of environments for any number of purposes. Thus, the claims set forth below are to be construed in view of the full breadth and spirit of the present disclosure as described herein.

What is claimed is:

1. A system, comprising:
a first terminal and a second terminal movable with respect to one another;
a sensor device operable to detect a change in capacitance between the first and second terminals as the first and second terminals move with respect to one another, wherein the sensor device comprises:
a two-stage charger connected with a controller and a voltage source, the two-stage charger comprising a first capacitor connected with a first switch and a second capacitor connected with a second switch, wherein the controller is operable to:
close the first switch to connect the first capacitor with the voltage source to charge the first capacitor; and
open the first switch and close the second switch to connect the second capacitor with the voltage source to charge the second capacitor.

2. The system of claim 1, wherein the controller is operable to equalize a first voltage of the first capacitor and a second voltage of the second capacitor for each cycle.

3. The system of claim 1, wherein the controller is operable to continuously charge and discharge the second capacitor using a Kalman filter.

4. The system of claim 3, wherein the two-stage charger comprises a third switch, and wherein the controller is operable to open the third switch when the first capacitor is being charged and close the third switch when the first capacitor is being discharged.

5. The system of claim 1, further comprising a pumping mechanism including a moveable piston, wherein the first terminal is connected to the piston.

6. The system of claim 5, wherein the second terminal is a conductive element, wherein the conductive element is coupled to a dielectric material.

7. The system of claim 6, wherein the dielectric material is a printed circuit board.

8. The system of claim 6, wherein the conductive element has a varied shape such that capacitance between the conductive element and first terminal increases between a first end and a second end of the conductive element.

9. A linear volume shuttle pump, comprising:
a first terminal and a second terminal movable with respect to one another, wherein the first terminal is a part of a pump mechanism;
a sensor device operable to detect a change in capacitance between the first and second terminals as the first and second terminals move with respect to one another, wherein the sensor device comprises:
a two-stage charger connected with a controller and a voltage source, the two-stage charger comprising a first capacitor connected with a first switch and a second capacitor connected with a second switch, wherein the controller is operable to:
close the first switch to connect the first capacitor with the voltage source to charge the first capacitor; and
open the first switch and close the second switch to connect the second capacitor with the voltage source to charge the second capacitor.

10. The linear volume shuttle pump of claim 9, wherein the controller is operable to equalize a first voltage of the first capacitor and a second voltage of the second capacitor for each pumping cycle.

11. The linear volume shuttle pump of claim 9, wherein the controller is operable to continuously charge and discharge the second capacitor using a Kalman filter.

12. The linear volume shuttle pump of claim 11, wherein the two-stage charger comprises a third switch, and wherein the controller is operable to open the third switch when the first capacitor is being charged and close the third switch when the first capacitor is being discharged.

13. The linear volume shuttle pump of claim 9, wherein the pumping mechanism comprises a piston grip, wherein the first terminal is part of the piston grip.

14. The linear volume shuttle pump of claim 9, wherein the second terminal is a conductive element, and wherein the conductive element has a varied shape such that capacitance between the conductive element and first terminal increases between a first end and a second end of the conductive element.

15. A method, comprising:
positioning a first terminal adjacent a second terminal, wherein the first terminal and the second terminal are movable with respect to one another;
detecting a change in capacitance between the first terminal and the second terminal using a sensor device, wherein the sensor device comprises a two-stage charger connected with a controller and a voltage source;
charging, by the controller, a first capacitor by closing a first switch to connect the first capacitor with the voltage source; and
charging, by the controller, a second capacitor by opening the first switch and closing a second switch to connect the second capacitor with the voltage source.

16. The method of claim 15, further comprising equalizing, by the controller, a first voltage of the first capacitor and a second voltage of the second capacitor for each pumping cycle.

17. The method of claim 15, further comprising continuously charging and discharging the second capacitor using a Kalman filter.

18. The method of claim 17, wherein continuously charging and discharging the second capacitor comprises:
opening, by the controller, a third switch when the first capacitor is being charged; and
closing, by the controller, the third switch when the first capacitor is being discharged.

19. The method of claim 15, further comprising providing a pumping mechanism including a piston grip, wherein the first terminal is part of the piston grip.

20. The method of claim 15, further comprising varying a shape of the second terminal such that capacitance between the first and second terminals increases between a first end and a second end of the second terminal.
